SEQUENCE LISTING (8 sequences)

SEQ ID NO: 1 - DNA, 842 bp, Mus musculus
ctgagaggaagttttatctgtgcagcccttctctgaggatggacacttctcacactacaaagtcctgtttgctgattcttcttgtggccctactgtgtgcagaaagagctcagggactggagtgttaccagtgctatggagtcccatttgagacttcttgcccatcaattacctgcccctaccctgatggagtctgtgttactcaggaggcagcagttattgtggattctcaaacaaggaaagtaaagaacaatctttgcttacccatctgccctcctaatattgaaagtatggagatcctgggtactaaggtcaacgtgaagacttcctgttgccaggaagacctctgcaatgtagcagttcccaatggaggcagcacctggaccatggcaggggtgcttctgttcagcctgagctcagtcctcctgcagaccttgctctgatggtcctcccaatgacctccacccttgtccttttatcctcatgtgcaacaattcttcctggagccctctagtgatgaattatgagttatagaagctccaaggtgggagtagtgtgtgaaataccatgttttgcctttatagcccctgctgggtaggtaggtgctctaatcctctctagggctttcaagtctgtacttcctagaatgtcattttgttgtggattgctgctcatgaccctggaggcacacagccagcacagtgaagaggcagaattccaaggtattatgctatcaccatccacacataagtatctggggtcctgcaatgttcccacatgtatcctgaatgtccccctgttgagtccaataaaccctttgttctcccaaaaaaaaaaaaaaa

SEQ ID NO: 2 - protein, 134 aa, Mus musculus
MDTSHTTKSCLLILLVALLCAERAQGLECYQCYGVPFETSCPSITCPYPDGVCVTQEAAVIVDSQTRKVKNNLCLPICPPNIESMEILGTKVNVKTSCCQEDLCNVAVPNGGSTWTMAGVLLFSLSSVLLQTLL

SEQ ID NO: 3 - protein, 131 aa, Homo sapiens
MKIFLPVLLAALLGVERASSLMCFSCLNQKSNLYCLKPTICSDQDNYCVTVSASAGIGNLVTFGHSLSKTCSPACPIPEGVNVGVASMGISCCQSFLCNFSAADGGLRASVTLLGAGLLLSLLPALLRFGP

SEQ ID NO: 4 - protein, 140 aa, Homo sapiens
MLPAAMKGLGLALLAVLLCSAPAHGLWCQDCTLTTNSSHCTPKQCQPSDTVCASVRITDPSSSRKDHSVNKMCASSCDFVKRHFFSDYLMGFINSGILKVDVDCCEKDLCNGAAGAGHSPWALAGGLLLSLGPALLWAGP

SEQ ID NO: 5 - protein, 184 aa, Homo sapiens
MKALGAVLLALLLFGRPGRGQTQQEEEEEDEDHGPDDYDEEDEDEVEEEETNRLPGGRSRVLLRCYTCKSLPRDERCNLTQNCSHGQTCTTLIAHGNTESGLLTTHSTWCTDSCQPITKTVEGTQVTMTCCQSSLCNVPPWQSSRVQDPTGKGAGGPRGSSETVGAALLLNLLAGLGAMGARRP

SEQ ID NO: 6 - protein, 15 aa, Artificial Sequence
  Synthetic
KVEKIGEGTYGVVKK

SEQ ID NO: 7 - DNA, 34 bp, Artificial Sequence
  Synthetic
ttatgcggccgcatgggctgtgtgcaatgtaagg

SEQ ID NO: 8 - DNA, 32 bp, Artificial Sequence
  Synthetic
gcatgcatgcttacaggttttcaccaggttgg

(19) United States
(12) Patent Application Publication
(10) Pub. No.: US 2005/0260202 A1
(43) Pub. Date: Nov. 24, 2005

(54) Methods for producing proliferating muscle cells

(75) Inventors: Harold S. Bernstein, San Francisco, CA (US); James Bristow, San Francisco, CA (US)

Correspondence Address: Christine A. Lekutis;MEDLEN & CARROLL, LLP, Suite 350, 101 Howard Street, San Francisco, CA, 94105, US

(73) Assignee: The Regents of the University of California, Oakland, CA (US)

(21) Appl. No.: 11/084,591

(22) Filed: Mar. 17, 2005

Related U.S. Application Data
(60) Provisional application No. 60/554,798, filed on Mar. 19, 2004.

(51) Int. Cl. A61K 39/395; A61K 48/00

(52) U.S. Cl. 424/145.1; 514/44

(57) ABSTRACT

The present invention is related to compositions and methods for expanding cell populations suitable for use as cardiac or skeletal muscle grafts. In particular, the present invention provides methods for regulation of cell cycle withdrawal and myoblast fusion during myogenesis.

This application claims priority to U.S. Patent Application No. 60/554,798, filed on Mar. 19, 2004.

The invention was made in part with government support from the National Institutes of Health, Grants HL62174, HL66727, HD00850 and T32HL07544. As such, the Unites States government has certain rights in the invention.

### FIELD OF THE INVENTION

The present invention is related to compositions and methods for expanding cell populations suitable for use as cardiac or skeletal muscle grafts. In particular, the present invention provides methods for regulation of cell cycle withdrawal and myoblast fusion during myogenesis.

### BACKGROUND OF THE INVENTION

The term heart failure refers to a clinical syndrome resulting from a cardiac disorder that impairs the ability of the ventricle to fill with, or eject a sufficient amount of blood, leading to breathing difficulties, fatigue, fluid retention, and decreased exercise tolerance. In the United States, ischemic (inadequate oxygenation) cardiomyopathy from coronary artery disease is the most common cause of heart failure. In fact, approximately 1.5 million people in the U.S. suffer acute myocardial infarction (heart attack) each year, 500,000 of which are fatal (Morb Mortal Wkly Rep, 50:90, 2001). At the cellular level, myocardial insufficiency results from the cumulative death of cardiac myocytes and the severely limited ability of remaining cells to proliferate (Urbanek et al., Proc Natl Acad Sci USA, 100:10440-10445, 2003; and Oh et al., Proc Natl Acad Sci USA, 100:12313-12318, 2003). Indeed, prognosis directly correlates with viable tissue mass after infarct (Lenderink et al., Circulation, 92:1110-1116, 1995).

One promising treatment aimed at limiting the consequences of postinfarction cardiac dysfunction, is the transplantation of cells (myocytes or stem cells) into the damaged left ventricle. Ideally, the transplanted cells would increase the ventricular ejection fraction by participating in cardiac contractions. Large numbers of potentially contractile cells, however, are required for intramyocardial grafting, because many of the cells die shortly after injection, as a consequence of apoptosis and inflammatory processes. Adult cardiomyocytes cannot be used for myocardial grafting, because these cells are terminally differentiated and do not proliferate. For this reason, other cell types have been used as cell grafts in experimental studies conducted in animal models, as well as in limited human clinical trials. Although superior results have been obtained with allogeneic fetal cardiomyocytes, the use of these cells presents greater logistic, immunologic, and ethical problems, than does the use of autologous adult skeletal myocytes or stem cells. In addition, not all cells found within mixed populations of adult skeletal myocytes or stem cells are competent to engraft to form functional muscle tissue.

Thus, improved methods for identifying, isolating and expanding competent autologous myocytes or stem cells from biopsy material are required to advance this therapy.

### SUMMARY OF THE INVENTION

The present invention is related to compositions and methods for expanding cell populations suitable for use as cardiac or skeletal muscle grafts. In particular, the present invention provides methods for regulation of cell cycle withdrawal and myoblast fusion during myogenesis.

The present invention provides methods comprising: contacting at least one cell expressing Sca-1 with a Sca-1 antagonist under growth conditions to produce an expanded cell population; and administering the cell population to a subject to regenerate muscle. In some embodiments, the growth conditions comprise conditions suitable for sustaining cell proliferation in the absence of cell fusion, conditions suitable for retaining Myf5 expression, conditions suitable for inducing myogenin expression, and/or the growth conditions comprise conditions suitable for the transient elevation of Fyn activity. In some preferred embodiments, the Sca-1 antagonist comprises but is not limited to one or more of a PIPLC, an antibody combination, a Sca-1 antisense molecule, a Sca-1 RNAi, and a Sca-1 synthetic ligand. In some particularly preferred embodiments, the Sca-1-reactive antibody combination comprises an antibody produced by a D7 clone and an antibody produced by an E13-161.7 clone. Also provided are methods in which the at least one cell is a purified from a tissue sample selected from but not limited to blood, bone marrow, and skeletal muscle. In preferred embodiments, the at least one cell is derived from the subject and/or the subject is a mammal. In some embodiments, the administering is accomplished by a means selected from but not limited to trans-coronary artery catheter (TCAC), intra-venous (IV) injection and intra-muscular injection (IM). In some preferred embodiments, the subject is diagnosed with a cardiovascular disease selected from but not limited to atherosclerosis, ischemia, hypertension, restenosis, angina pectoris, rheumatic heart disease, congenital cardiovascular defect, and arterial inflammation, while in other preferred embodiments, the subject is diagnosed with a skeletal muscle injury or muscular degeneration. Moreover, the present invention provides methods comprising: contacting at least one cell expressing Sca-1 with a Sca-1 antagonist under growth conditions to produce an expanded cell population; and administering the cell population to a subject to study the regeneration of muscle.

In addition, the present invention provides compositions comprising an expanded cell population produced by contacting at least one cell expressing Sca-1 with a Sca-1 antagonist under growth conditions, and a buffer, wherein the growth conditions comprise conditions suitable for sustaining cell proliferation in the absence of cell fusion. In some embodiments, the expanded cell population comprises at least 1×10⁵ cells, while in other embodiments, the expanded cell population comprises from 1×10³ to 1×10⁹ cells. In some preferred embodiments, a majority of cells of the expanded cell population express one or more muscle differentiation antigens selected from but not limited to Myf5, MyoD, and Myogenin. In particularly preferred embodiments, the majority of cells comprise at least 50%, at least 75%, or at least 90% of the expanded cell population.

### BRIEF DESCRIPTION OF FIGURES

FIG. 1 illustrates that treatment with PIPLC strips Sca-1 from the surface of C2C12 cells as analyzed by immunoblot in panel A, and by immunostaining in panel B.

FIG. 2 illustrates that treatment with PIPLC inhibits cell cycle withdrawal and myotube formation in rodent and avian myoblasts. Mouse C2C12 myoblasts (panels A-C), mouse Sol8 myoblasts (Panels D-F), rat L6 myoblasts (panel G-I), and quail QM7 myoblasts (panels J-L) were grown under differentiation conditions in the presence and absence of PIPLC (5 U/ml) for 5, 7, or 10 days, respectively, and stained with DAPI and labeled with BrdU. The bar in the phase contrast images is 50 uM in length. Data are shown as the mean+/−SEM for N=5.

FIG. 3 illustrates that inhibition of Sca-1 by treatment with either an anti-Sca-1 monoclonal antibody combination (panels A-C) or with Sca-1 antisense expression (panels D-F) reduces myoblast cell cycle withdrawal and fusion under differentiation conditions. The data in panels B and C are shown as the mean+/−SEM for N=3, while the data in panels E and F are shown as the mean+/−SEM for N=4.

FIG. 4 illustrates that Sca-1 downregulation by antisense expression blocks myogenesis as determined by immunostaining under differentiation conditions. As depicted in panel A, both proliferating (Pro) control (C) cells and Sca-1 antisense (AS) cells expressed Myf5, MyoD, and myogenin, whereas under differentiation conditions (day 5) Myf5 expression persisted in antisense but not control cells. As depicted in panel B, control cells were Myf5-negative and BrdU-negative after culture for 5 days in differentiation medium, while Sca-1 antisense cells were positive for both Myf5 and BrdU when cultured under the same conditions.

FIG. 5 illustrates that Fyn mediates Sca-1 function during myogenesis in vitro. As depicted in the graphs of panel A, control C2C12 cells grown in differentiation medium for 2 and 5 days have increased Fyn activity, whereas Src activity in these cells remains low. By comparison, Sca-1 antisense cells grown in differentiation medium have an inappropriate increase in Fyn activity on day 2. As depicted in the graph of panel B, myoblast fusion was elevated in control cells transfected with a constitutively-active Fyn mutant (Y531F), while conversely, myoblast fusion was reduced in Sca-1 antisense cells transfected with a dominant-negative Fyn mutant (K299M). In both panels, data are shown as the mean+/−SEM for N=3.

FIG. 6 provides the DNA and protein sequences of murine Sca-1. The nucleic acid sequence of murine Sca-1 is shown in panel A (GENBANK Accession No. NM_{—}010738, disclosed as SEQ ID NO:1). The coding region extends from nucleotides 39 to 443. Panel B provides an alignment of the amino acid sequence of murine Sca-1 (GENBANK Accession No. NP_{—}034868, disclosed as SEQ ID NO:2), with human Ly-6 family members RIG-E (GENBANK Accession No. NP_{—}002337, disclosed as SEQ ID NO:3), Ly-6H (GENBANK Accession No. NP_{—}002338, disclosed as SEQ ID NO:4), and HDL-BP (GENBANK Accession No. AAH63857, disclosed as SEQ ID NO:5).

FIG. 7 provides a western blot illustrating that the beta1-integrin associates with Sca-1 in lysates of differentiating C2C12 myoblasts.

FIG. 8 provides FACS histograms and phase contrast images of freshly isolated and in vitro-differentiated mouse skeletal myoblasts. Flow cytometric analysis of freshly isolated myoblasts stained with desmin, MyoD and myosin antibodies is depicted in panel A, while panel B depicts the alpha-7 integrin (a known marker for myoblasts) staining before and after sorting on the basis of alpha-7 integrin expression. Panel C provides phase contrast images of freshly isolated and cultured myoblasts. The black arrows denote multinucleated myotubes.

FIG. 9 provides a FACS histogram of freshly harvested mouse cardiac myocytes stained with Sca-1 and myosin antibodies.

FIG. 10 illustrates that Sca-1 is expressed on a subpopulation of C2C12 cells and is removed by PIPLC. Panel A is a flow histogram of proliferating C2C12 cells (Pro) and cells grown in differentiation media for two days (Day 2) demonstrating that the percentage of cells with surface expression of Sca-1 increases during differentiation, but that Sca-1-positive and −negative populations persist. Gates were established by nonspecific PE-conjugated antibody binding in each experiment. A representative profile is shown with mean values for percent Sca-1-positive and −negative cells given (n=3; *significant difference, P<0.05). Panel B is a flow histogram showing a decrease in the subpopulation of Sca-1-expressing C2C12 cells in cultures grown for 2 days in differentiation medium (Day 2) and stably transfected with Sca-1 antisense (AS), compared with cells transfected with an empty vector (S). The asterisk indicates a significant difference of P=0.035. There was a small, but not statistically significant, decrease in the percent Sca-1-expressing cells in proliferating cultures (Pro) stably transfected with Sca-1 antisense (AS), compared with cells transfected with empty vector (S), P=0.4. Gates were established by nonspecific PE-conjugated antibody binding in each experiment. A representative profile is shown with mean values for percent Sca-1-positive and −negative cells given (n=3).

FIG. 11 illustrates that Sca-1 is expressed on a subpopulation of primary murine skeletal myoblasts. Flow cytometry of primary myoblasts isolated from the hind limbs of C57BL/6 mice and cultured in differentiation medium (DM) for at least 3 days demonstrated that a subpopulation of Sca-1-positive cells and small myotubes, constituting 25% of selected myoblasts at the time of isolation, decreased in number with differentiation and expression of myosin as shown in panel A. Sca-1 expression in this residual population of Sca-1 expressors increased on a per cell basis (panel B), whereas the size of these cells, as a function of forward angle light scatter (FALS), remained constant (panel C). All data points are shown, and trendlines were generated by bivariate regression analysis using the Pearson coefficient with significance levels indicated.

### DEFINITIONS

To facilitate an understanding of the present invention, a number of terms and phrases are defined below:

As used herein, the term “gene” refers to a nucleic acid (e.g., DNA) sequence that comprises coding sequences necessary for the production of a polypeptide or precursor or RNA (e.g., tRNA, siRNA, rRNA, etc.). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends, such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

In particular, the term “Sca-1 gene” refers to the full-length Sca-1 nucleotide sequence. However, it is also intended that the term encompass fragments of the Sca-1 nucleotide sequence, as well as other domains (e.g., functional domains) within the full-length Sca-1 nucleotide sequence. Furthermore, the terms “Sca-1 gene,” “Sca-1 nucleotide sequence,” and “Sca-1 polynucleotide sequence” encompass DNA, cDNA, and RNA sequences. The terms “Sca-1” “stem cell antigen-1” “Ly-6A” “Ly-6E” and “TAP” as used herein, refer to a murine Ly-6 gene (e.g., Mus musculus GENBANK Accession No. NM_{—}010738) and its gene product, as well as its vertebrate counterparts, including wild type and mutant products. In some preferred embodiments, the term Sca-1 refers to a human homolog of murine Sca-1.

As used herein, the term “proliferation” refers to an increase in cell number by division. Proliferation can be measured by methods known in the art such as bromodeoxyuridine or triated thymydine incorporation.

As used herein, the phrases “cell fusion” refers to the joining of two previously separate cells, which occurs naturally during the formation of vertebrate skeletal muscle.

As used herein, the term “plasmid” refers to a small, independently replicating, piece of DNA. Similarly, the term “naked plasmid” refers to plasmid DNA devoid of extraneous material typically used to affect transfection. As used herein, a “naked plasmid” refers to a plasmid substantially free of calcium-phosphate, DEAE-dextran, liposomes, and/or polyamines.

As used herein, the term “purified” refers to molecules (polynucleotides or polypeptides) that are removed from their natural environment (and/or contaminants), isolated or separated. “Substantially purified” molecules are at least 50% free, preferably at least 75% free, and more preferably at least 90% free from other components with which they are naturally associated.

As used herein, the term “recombinant DNA” refers to a DNA molecule that is comprised of segments of DNA joined together by means of molecular biology techniques. Similarly, the term “recombinant protein” refers to a protein molecule that is expressed from recombinant DNA.

As used herein, the term “portion” when used in reference to a protein (as in “a portion of a given protein”) refers to fragments of that protein. The fragments may range in size from four amino acid residues to the entire amino acid sequence minus one amino acid. Similarly, when used in reference to cells, the term “portion” (as in “a portion of the cells”) refers to any amount less than the total number of cells available. When the term “amino acid sequence” is recited herein, to refer to an amino acid sequence of a naturally occurring protein molecule, “amino acid sequence” and like terms (such as “polypeptide” or “protein”) are not meant to limit the amino acid sequence to the complete, native amino acid sequence associated with the recited protein molecule.

As used herein, the term “wild-type” refers to a gene or gene product that has the characteristics of that gene or gene product when isolated from a naturally occurring source. A wild type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally occurring mutants can be isolated; these are identified by the fact that they have altered characteristics when compared to the wild-type gene or gene product.

As used herein, the term “staining” refers to any number of processes known to those in the field that are used to better visualize, distinguish or identify a specific component(s) and/or feature(s) of a cell or cells. In some preferred embodiments, the term staining refers to immunofluorescence analysis. The term “immunofluorescence analysis” refers to a staining technique used to identify, mark, label, visualize or make readily apparent by procedures known to those practiced in the art, where a ligand (usually an antibody) is bound to a receptor (usually an antigen) and such ligand, if an antibody, is conjugated to a fluorescent molecule, or the ligand is then bound by an antibody specific for the ligand, and the antibody is conjugated to a fluorescent molecule, where said fluorescent molecule can be visualized with the appropriate instrument (e.g., fluorescent microscope, FACS, etc.).

As used herein the term “antigen” refers to a protein, glycoprotein, lipoprotein, lipid or other substance that is reactive with an antibody specific for a portion of the molecule.

As used herein, the terms “mammal” and “mammalian” refer animals of the class mammalia, which nourish their young by fluid secreted from mammary glands of the mother, including human beings (e.g., mice, rats, pigs, monkeys, humans, etc.). The term “subject” shall be defined as a human or other animal, such as a guinea pig or mouse and the like, capable of donating and receiving myoblasts. Typically, the terms “subject” and “patient” are used interchangeably.

As used herein, the term “control” refers to subjects or samples that provide a basis for comparison for experimental subjects or samples. For instance, the use of control subjects or samples permits determinations to be made regarding the efficacy of experimental procedures. In some embodiments, the term “control subject” refers to animals that receive a mock treatment (e.g., PBS alone).

As used herein, the terms “Sca-1 antagonist” and “Sca-1 inhibitor” refer to any molecule that reduces the expression of or activity of Sca-1. Sca-1 antagonists suitable for use in the methods and compositions of the present invention include but are not limited to antisense molecules, RNAi molecules, Sca-1-reactive antibody combinations, dominant negative mutants, artificial ligands and PIPLC.

As used herein, the term “antisense molecule” refers to polynucleotides and oligonucleotides capable of binding to an mRNA molecule. In particular, an antisense molecule is a DNA or RNA sequence complementary to an mRNA sequence of interest. In preferred embodiments, the term Sca-1 antisense molecule refers to a single-stranded DNA or RNA sequence that binds to at least a portion of a Sca-1 mRNA molecule to form a duplex which then blocks further transcription and/or translation.

As used herein, the term “RNAi” refers to a double stranded RNA molecule, with each strand consisting of at least 20 nucleotides that directs the sequence-specific degradation of mRNA through a process known as RNA interference. Thus RNAI can be used to block gene expression posttranscriptionally (Zamore et al., Cell, 101:25-33, 2000).

As used herein, the term “antibody” refers to polyclonal and monoclonal antibodies. Polyclonal antibodies which are formed in the animal as the result of an immunological reaction against a protein of interest or a fragment thereof, can then be readily isolated from the blood using well-known methods and purified by column chromatography, for example. Monoclonal antibodies can also be prepared using known methods (See, e.g., Winter and Milstein, Nature, 349:293-299, 1991). As used herein, the term “antibody” also encompasses recombinantly prepared, and modified antibodies and antigen-binding fragments thereof, such as chimeric antibodies, humanized antibodies, multifunctional antibodies, bispecific or oligo-specific antibodies, single-stranded antibodies and F(ab) or F(ab)₂ fragments. The term “reactive” in used in reference to an antibody indicates that the antibody is capable of binding an antigen of interest. For example, a Sca-1-reactive antibody is an antibody that binds to Sca-1 or to a fragment of Sca-1.

As used herein, the term “dominant negative mutant” refers to molecules that lack wild type activity, but which effectively compete with wild type molecules for substrates, receptors, etc., and thereby inhibit the activity of the wild type molecule. In preferred embodiments, the term “Sca-1 dominant negative mutant” refers to a Sca-1 mutant protein that competes with the wild type Sca-1 protein for ligands or adaptor molecules, but which fails to induce downstream effects.

As used herein, the terms “GPI-PLC” and “(glycosyl)phosphatidylinositol-specific phospholipase C” refer to an enzyme that hydrolyzes glycerophosphatidates with the formation of 1,2-diacylglycerol and a phosphorylated nitrogenous base (e.g., 6-(a-D-glucosaminyl)-1-phosphatidyl-1D-myo-inositol=6-(a-D-glucosaminyl)-1D-myo-inositol 1,2-cyclic phosphate+1,2-diacyl-sn-glycerol). Other names for GPI-PLC include, GPIPLC, GPI-specific phospholipase C, VSG-lipase, glycosyl inositol phospholipid anchor-hydrolyzing enzyme, glycosylphosphatidylinositol-phospholipase C, glycosylphosphatidylinositol-specific phospholipase C, and variant-surface-glycoprotein phospholipase C.

As used herein, the term “myoblast” refers to a muscle cell that has not fused with other myoblasts to form a myofibril and has not fused with an existing myofibril.

As used herein, the term “cardiovascular disease” refers to any disease affecting the heart or blood vessels, and in general refers to vascular diseases caused by atherosclerosis.

The term “atherosclerosis” refers to the progressive narrowing and hardening of the arteries over time. This is known to occur to some degree with aging, but risk factors such as high cholesterol, high blood pressure, smoking, diabetes, and a family history of atherosclerotic disease, accelerates this process.

The term “myocardial ischemia” refers to a lack of adequate oxygenation of the heart. The low oxygen state is usually due to obstruction of the arterial blood supply or inadequate blood flow leading to hypoxia in the tissue.

The term “hypertension” refers to persistently high arterial blood pressure. Hypertension may have no known cause (essential or idiopathic hypertension) or may be associated with other primary diseases (secondary hypertension).

The term “restenosis” refers to a recurrence of stenosis after corrective surgery on the heart. In other words “restenosis” is a narrowing of a structure (usually a coronary artery) following the removal or reduction of a previous narrowing.

The term “angina pectoris” refers to a heart condition marked by spasmatic (paroxysmal) chest pain due to reduced oxygenation of the heart.

The term “rhematic heart disease” refers to a disease involving inflammation of and damage to heart valves following streptococcal infection.

The term “congenital cardiovascular defect” refers to structural abnormalities of the cardiovascular system present at birth. Most heart defects either obstruct blood flow in the heart or vessels near it, or cause blood to flow through the heart in an abnormal pattern. The term “congenital cardiovascular defect” includes but is not limited to aortic stenosis, atrial septal defect, atrioventricular canal defect, bicuspid aortic valve, coarctation of the aorta (Coarct), Ebstein's anomaly, Eisenmenger's complex, hypoplastic left heart syndrome, patent ductus arteriosus, pulmonary stenosis, pulmonary atresia, subaortic stenosis, tetralogy of Fallot, total anomalous pulmonary venous connection, transposition of the great arteries, tricuspid atresia, truncus arteriosus, ventricular septal defect, and patent ductus arteriosus.

The term “arterial inflammation” refers to an immune response occurring in arteries as a result of injury, infection or irritation.

The phrase “diagnosed with cardiovascular disease” refers to a diagnosis made by a physician including one or more of a general examination, blood tests (e.g., cholesterol level, oxygen content, etc.), chest X-ray and an electrocardiogram (EKG). An “electrocardiogram” is a painless recording of the heart's electrical activity, detected by small metal electrodes, which are placed on the patient's wrists, ankles and chest.

As used herein, the term “skeletal muscle injury” refers damage or trauma to the striated muscle of a subject (muscle under voluntary control).

As used herein, the term “muscular degeneration” refers to the impairment or worsening of the function of muscle. In contrast, the term “muscular regeneration” refers to the growth of new muscle.

The term “muscular dystrophy” refers to a group of diseases characterised by progressive degeneration and/or loss of muscle fibres without nervous system involvement, most of which have a hereditary origin. The details of the type of genetic defect and of the prognosis for the disease vary from type to type. Duchenne muscular dystrophy (pseudohypertrophic muscular dystrophy) is the most common form, due to a sex-linked recessive allele manifested by an absence of the protein dystrophin.

As used herein, the terms “transplant cells” and “graft material” refer broadly to cells being grafted, implanted or transplanted. The term “transplantation” refers to the transfer or grafting of cells from one part of a subject to another part of the same subject, or to another subject. As used herein, a transplant cells may comprise a collection of cells of identical or similar composition, or derived from an organism (i.e., a donor), or from an in vitro culture (i.e., a tissue culture system). The term “suitable graft material” refers to cells with the desired phenotype (e.g., proliferation competent, unfused, and/or expressing a muscle differentiation antigen), and which are free of deleterious contaminants (e.g., free of bacteria and fungi).

As used herein, the term “buffer” refers to an ionic compound that resists changes in its pH. Preferred embodiments of the present invention comprise isotonic buffers (e.g., physiologic salt solution). The term “isotonic” refers to a solution in which body cells can be bathed without a net flow of water across the semipermeable cell membrane.

### DESCRIPTION OF THE INVENTION

Myogenesis, the process of muscle cell determination, differentiation, and fusion into multinucleated syncitia, is essential for normal muscle development and tissue regeneration following injury. Much is known about the transcriptional regulation of myogenesis. The myogenic regulatory factors MyoD, Myf-5, myogenin, and MRF4 act to initiate and maintain the differentiated state (Puri and Sartorelli, J Cell Physiol, 185:155-173, 2000). Additionally, they inhibit cell cycle progression by upregulating expression of the retinoblastoma protein and members of the Cip1/Kip1 family of cyclin-dependent kinase inhibitors, and downregulating the expression of G1 effectors cyclin D1 and cyclin-dependent kinase 4 (Walsh and Perlman, Curr Opin Genet Dev, 7:597-602, 1997).

While signaling events during myocyte differentiation have been studied in detail, cell surface molecules that transmit signals from the extracellular milieu to these intracellular pathways are less well understood. Recent genetic studies in Drosophila have provided the first insights into mechanisms of myoblast determination and fusion. The immunoglobulin (Ig)-like proteins Dumbfounded and Sticks and Stones support cellular adherence, while the membrane- and cytoskeletal-associated proteins Myoblast City and Rolling Stone facilitate formation of the fusion complex, which also requires cytoplasmic signaling proteins Rolling Pebbles and Antisocial (Taylor, Current Biology, 12:R224-R228, 2002).

Other cell surface molecules have been linked to muscle differentiation in vertebrates. Antibody blockade of ligand binding to β1 integrins in chick embryos results in a predominance of early myogenic precursors (Menko and Boettiger, Cell, 51:51-57, 1987), and while β1 integrin-null myoblasts have been shown to be capable of fusion (Hirsch et al., J Cell Sci, 111:2397-2409, 1998) mice lacking β1 integrin specifically in muscle have poorly developed muscle fibers (Schwander et al., Dev Cell, 4:673-685, 2003). In contrast, α4 integrin-deficient murine embryonic stem cells can be differentiated into myoblasts that fuse to form myotubes (Yang et al., J Cell Biol, 135:829-835, 1996). Antibody interference of N-cadherin during early chick embryogenesis disrupts formation of early myogenic precursors (George-Weinstein et al., Dev Biol, 185:14-24, 1997), and antibodies against N-cadherin inhibit cellular adherence in cultured chick myoblasts (Knudsen et al., Exp Cell Res, 188:175-184, 1990), however, N-cadherin null embryonic stem cells can differentiate into myoblasts with normal fusion properties (Charlton et al., J Cell Biol, 138:331-336, 1997).

I. Role of Sca-1 in Myoblast Proliferation and Fusion

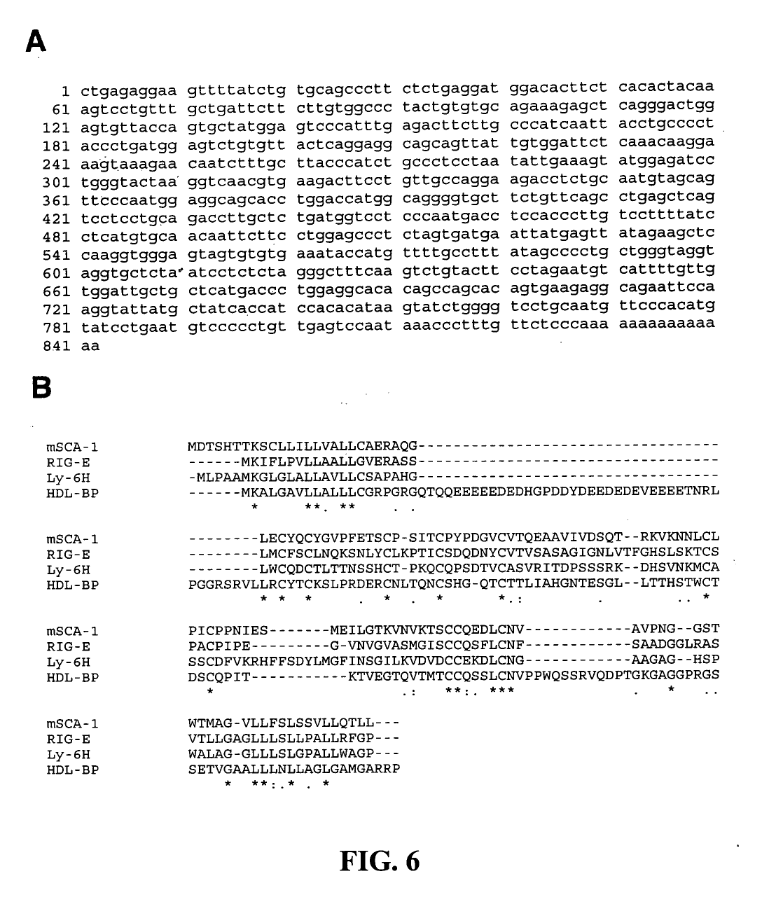

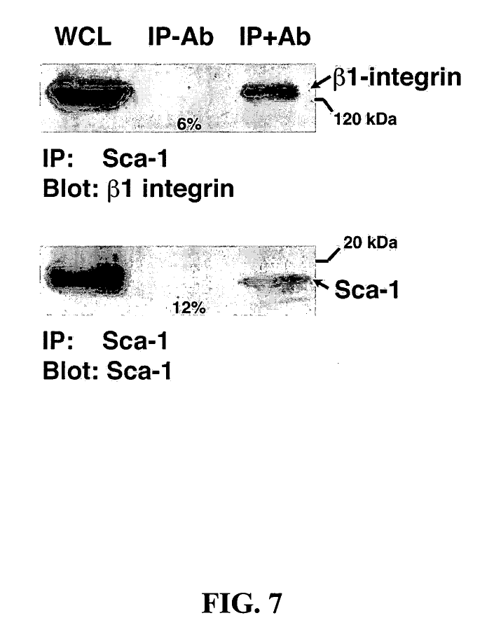

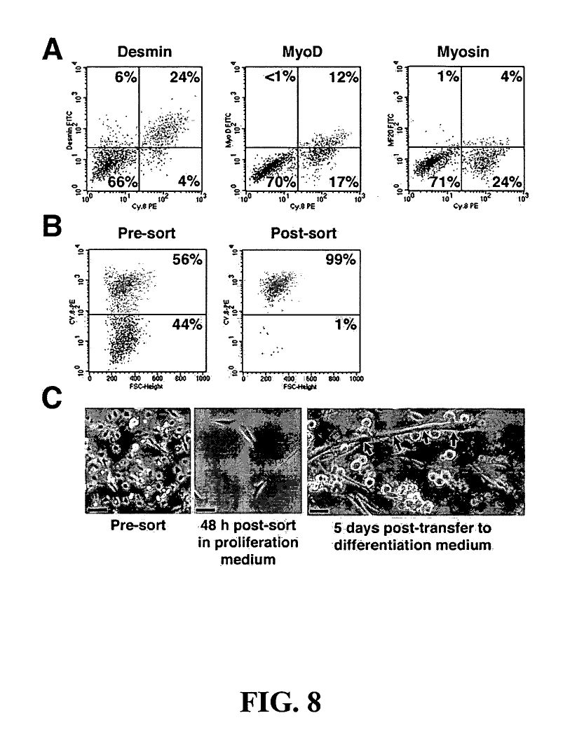

Sca-1 is a member of the murine Ly-6 multigene family encoding a number of highly homologous, glycosyl-phosphatidylinositol (GPI)-anchored membrane proteins, expressed on hematopoietic and lymphoid cells. Homologs of various murine Ly-6 molecules have been identified in multiple species. The most well characterized family member is Sca-1/Ly-6A, an antigen commonly used for purification of murine pluripotent hematopoietic cells (Patterson et al., Blood, 95:3125-3132, 2000). To date, several human GPI-anchored Ly-6 molecules have been cloned, the most similar to murine Sca-1 (mSca-1) include: Sca-2/Tsa-1/RIG-E, Ly-6H, and HDL-BP (FIG. 6). Although the ligand for Sca-1 is not known, studies have implicated Sca-1 and related family members in cell-cell adhesion among thymocytes (Bamezai and Rock, Proc Natl Acad Sci USA, 92:4294-4298, 1995; and Classon and Boyd, Dev Immunol, 6:149-156, 1998). Interestingly, ligands for other superfamily members contain EGF-like repeats, and a ligand for Ly-6D demonstrates significant homology to the Notch family responsible for somatic patterning in Drosophila (Aposolopoulos et al., Immunity, 12:223-232, 2000). More recently, Sca-1 has been reported as a marker for muscle-derived stem cells (Asakura et al., J Cell Biol, 159:123-134, 2002; and Jankowski et al., Hum Gene Ther, 12:619-628, 2001), and Sca-1-positive myogenic precursor cells appear to be recruited from the circulation to sites of muscle injury (Oh et al., Proc Natl Acad Sci USA, 100:12313-12318, 2003). A specific function for the Sca-1 antigen in muscle development, however, has not been described.

During a screen for regulators of myoblast differentiation, the inventors determined that expression of the stem cell marker, Stem cell antigen-1 (Sca-1/Ly-6A), was transiently upregulated during myocyte cell cycle withdrawal (Shen et al., Dev Dyn, 226:128-138, 2003; herein incorporated by reference in its entirety). Briefly, the inventors had determined that Sca-1 expression was markedly increased coincident with the time point at which cells expressed the first markers of both cell cycle withdrawal and commitment to myocyte differentiation. In contrast, Sca-1 transcript was downregulated in proliferating myoblasts and differentiated myotubes relative to this intermediate time point.

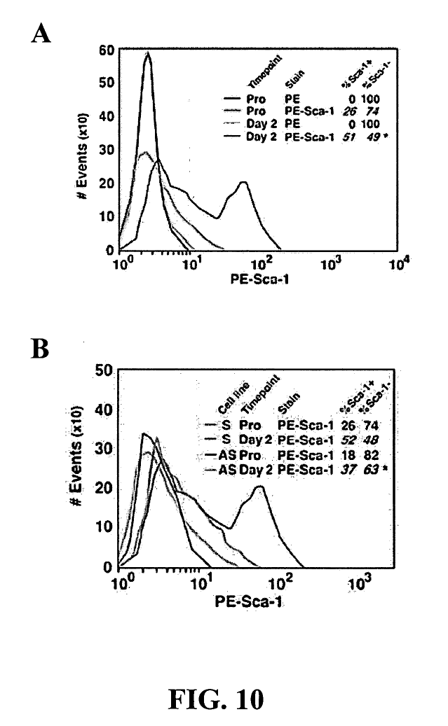

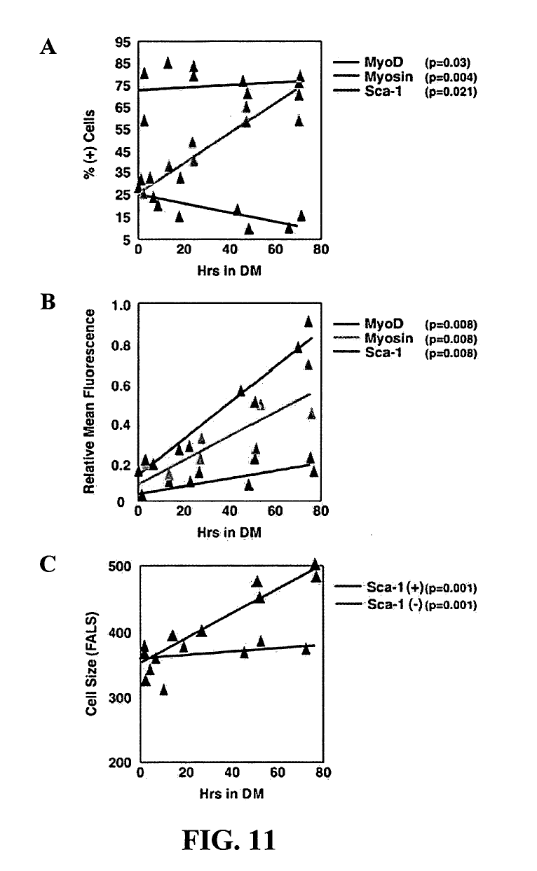

Now, during development of the present invention, a previously unrecognized role for Sca-1 in myoblast proliferation and fusion was identified. In the first place, the level of Sca-1 expressed on the surface of C2C12 myoblasts on a per cell basis was examined by flow cytometryon proliferating C2C12 cells, and cells grown in differentiation medium for two days, which is the time point of maximal Sca-1 expression (FIG. 10A). Whereas the majority (74%) of proliferating myoblasts were Sca-1-negative, more than half of the cells (51%) were positive for Sca-1 surface expression at the time of cell-cycle withdrawal during myogenesis (P=0.008). Although immunoblot analysis suggested that Sca-1 expression may decline after five days in differentiation medium, the heterogeneity of cell size in differentiated cultures precluded their analysis by flow cytometry. However, these data indicate that subpopulations of C2C12 cells (Sca-1-positive and Sca-1-negative) clearly exist. Similarly, Sca-1 was found to be expressed on a subpopulation of primary murine skeletal myoblasts, with this population decreasing in number with myogenic differentiation and the expression of myosin (FIG. 11A-C).

As described in more detail in the experimental examples, stripping Sca-1 from the extracellular surface, blocking Sca-1 with antibodies, or downregulating Sca-1 expression by antisense all produced a similar striking phenotype, that of sustained proliferation and interference with myoblast fusion coincident with the expression of early markers of myogenic differentiation. In addition, antisense-mediated downregulation of Sca-1 also derepressed Fyn kinase activity precisely during that time when myoblasts withdraw from the cell cycle and begin to fuse, and Fyn mutants recapitulated and rescued the Sca-1-antisense phenotype.

The role of Sca-1 in myoblast differentiation and cell cycle withdrawal is contemplated to be analogous to its role in T cells, where the absence of Sca-1 results in a more rapid and prolonged T cell proliferative response (Stanford et al., J Exp Med, 186:705-717, 1997). It is also contemplated that Fyn activity is temporally regulated during myoblast fusion by Sca-1. Normally, Fyn activity is upregulated in differentiated myotubes, and is necessary for protecting post-mitotic muscle cells from apoptosis (Laprise et al., J Cell Physiol, 191:69-81, 2002). Downregulation of Sca-1 by, for example, antisense leads to premature activation of Fyn, which is contemplated to be responsible for sustained myoblast proliferation, indicating that Sca-1 plays a role in either suppressing Fyn activity or influencing the integration of Fyn with other signaling pathways that converge on the early myogenic program.

While a role for Fyn in cellular proliferation and survival has been established in a variety of cell types (Resh, Int J Biochem Cell Bio, 30:1159-1162, 1998), how Sca-1 couples to Fyn has not been determined. Plasma membrane microdomains couple signaling in space and time between GPI-anchored proteins on the extracellular surface and signaling proteins compartmentalized in the cytoplasmic leaflet of the plasma membrane, such as nonreceptor protein tyrosine kinases and G proteins (Alonso and Millan, J Cell Sci, 114:3957-3965, 2001; Matko and Szollosi, Immunol Lett, 82:3-15, 2002). Transmembrane-spanning co-receptors also coalesce in these microdomains, and may be required for transmitting signals from GPI-anchored proteins (Werlen and Palmer, Curr Opin Immunol, 14:229-305, 2002). Thus, it is contemplated that Sca-1 either signals directly through Fyn, or brings components of membrane microdomains together to regulate Fyn activation, although an understanding of the mechanism is not necessary to practice the present invention and the present invention is not limited to any particular mechanism

Sca-1 null mice have been reported as grossly normal, with T-lymphocytes having a more rapid and prolonged proliferative response (Stanford et al., supra, 1997). Whether these mice have abnormal muscle phenotypes with respect to performance or regenerative capacity is not known. That said, several recent studies have implicated Sca-1 as a marker of myogenic precursor cells that are recruited during regeneration following muscle injury (Jankowski et al., Hum Gene Ther, 12:619-628, 2001; LaBarge and Blau, Cell, 111:589-601, 2002; Oh et al., Proc Natl Acad Sci USA, 100:12313-12318, 2003; and Polesskaya et al., Cell, 113:841-852, 2003).

Mice null for Thymic Shared Antigen-1, another Ly-6 superfamily member, recently have been reported to exhibit growth delay at E14.5 and absorption by E16 (Zammit et al., Mol Cell Biol, 22:946-952, 2002). This was preceded by the appearance of thinned ventricular myocardium with abnormal trabeculation, in the presence of otherwise normal atrioventricular and semilunar valves, at E14. Thus, it is contemplated that the TSA-1 knock-out mice have a primary defect in myocardial development, consistent with an important role for Ly-6 family members in muscle development. Even so, an understanding of any of the mechanism(s) involved is not necessary in order to make or use the present invention, and it is not intended that the present invention be limited to any particular mechanism(s).

II. Sca-1 Antagonists

The present invention provides methods comprising contacting at least one Sca-1+ cell with one or more Sca-1 antagonists or inhibitors. In preferred embodiments, the Sca-1+ cell is grown in culture in the presence of a Sca-1 antagonist to produce an expanded population of cells. In particularly preferred embodiments, the expanded cell population is suitable for the regeneration of cardiac or skeletal muscle in vivo, and/or for in vitro or in vivo use to screen for compounds that regulate regeneration, differentiation, or related processes. Although, specific Sca-1 antagonists are disclosed in the experimental examples, the invention is not limited to the use of these compounds. In fact, additional Sca-1 antagonists are also contemplated to be suitable for expanding cell populations suitable for muscle regeneration.

In particular, additional Sca-1 reactive antibodies can be prepared using various immunogens. In one embodiment, the immunogen is a human Sca-1 homolog. Antibodies contemplated to be suitable for use with the present invention include, but are not limited to polyclonal, monoclonal, chimeric, single chain, Fab fragments, and Fab expression libraries.

Various procedures known in the art may be used for the production of polyclonal antibodies directed against Sca-1. For the production of antibodies, various host animals including but not limited to rabbits, mice, rats, sheep, and goats, can be immunized by injection with a peptide corresponding to one or more Sca-1 epitopes. In a preferred embodiment, the peptide is conjugated to an immunogenic carrier (e.g., diphtheria toxoid, bovine serum albumin, keyhole limpet hemocyanin, etc.). Various adjuvants may be used to increase the immunological response, depending on the host species, including but not limited to Freund's (complete and incomplete), mineral gels (e.g., aluminum hydroxide), surface-active substances (e.g., lysolecithin, pluronic polyols, polyanions, peptides, oil emulsions, keyhole limpet hemocyanins, dinitrophenol, and potentially useful human adjuvants such as Bacille Calmette-Guerin and Corynebacterium parvum).

For preparation of additional monoclonal antibodies directed toward Sca-1, it is contemplated that any technique that provides for the production of antibody molecules by continuous cell lines in culture will find use with the present invention (See e.g., Harlow and Lane, Antibodies: A Laboratory Manual, Cold Spring Harbor Laboratory Press, Cold Spring Harbor, N.Y.). These include but are not limited to the hybridoma technique originally developed by Köhler and Milstein (Köhler and Milstein, Nature, 256:495-497, 1975), as well as the trioma technique, the human B-cell hybridoma technique, and the EBV-hybridoma technique to produce human monoclonal antibodies.

III. Compositions for Cell Transplantation

The present invention provides pharmaceutical compositions suitable for treating and studying heart disease and skeletal muscle injury. In particular, the present invention provides pharmaceutical compositions comprising Sca-1+ cell populations expanded in the presence of one or more Sca-1 antagonists. The expanded cell populations of the present invention are administered in any sterile, biocompatible solution including but not limited to saline, phosphate buffered saline, Hanks' solution, and Ringer's solution. In preferred embodiments, the biocompatible solution is an isotonic solution.

Pharmaceutical compositions suitable for use in the present invention include compositions wherein the active ingredients are contained in an effective amount to achieve the intended purpose. For example, an effective amount of the expanded cells may be that amount suitable for improving ventricular function after myocardial infarction. Determination of effective amounts is well within the capability of those skilled in the art, especially in light of the disclosure provided herein. To begin with, the therapeutically effective dose of expanded cells is estimated initially from cell culture assays. Then, preferably, dosage is formulated in animal models (particularly murine models) to achieve the desired therapeutic result. The exact dosage is chosen by the individual health care provider (e.g., physician or veterinarian) in view of the subject to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Normal dosage amounts may vary from 1×10³ to 1×10⁹ expanded cells, depending upon the route of administration (e.g., intramuscular, intravenous, intraarterial, intraventricular, etc.).

Experimental

The following examples are provided in order to demonstrate and further illustrate certain preferred embodiments and aspects of the present invention and are not to be construed as limiting the scope thereof.

In the experimental disclosure which follows, the following abbreviations apply: eq (equivalents); M (Molar); mM (millimolar); μM (micromolar); N (Normal); mol (moles); mmol (millimoles); μmol (micromoles); nmol (nanomoles); g (grams); mg (milligrams); μg (micrograms); ng (nanogramso); l or L (liters); ml (milliliters); μl (microliters); cc (cubic centimeters); cm (centimeters); mm (millimeters); μm (micrometers); nm (nanometers); ° C. (degrees Centigrade); bp (base pair); kb (kilobase); PCR (polymerase chain reaction); cpm (counts per minute); BrdU (bromodeoxyuridine); SDS (sodium dodecyl sulfate); MgCl₂ (magnesium chloride); NaCl (sodium chloride); DAPI (4′,6-diamidino-2-phenylindole); EDTA (ethylene diamine tetraacetic acid); BSA (bovine serum albumin); FBS (fetal bovine serum); DMEM (Dulbecco's Modified Eagle's medium); GPI (glycosyl-phosphatidylinositol); PIPLC (phosphatidylinositol-specific phospholipase-C); mAb (monoclonal antibody); PBS (phosphate buffered saline); Tris (tris(hydroxymethyl)aminomethane); H₂O (water); and IgG (immunoglobulin).

In addition, reagents and materials have been or are obtained from the following sources: ATCC (American Type Culture Collection, Manassus, Va.); Invitrogen (Invitrogen Life Technologies, Carlsbad, Calif.); Roche (F. Hoffmann-La Roche Limited, Basel, Switerland); Sigma (Sigma Chemical Co., St. Louis, Mo.); Jackson (Jackson Laboratory, Bar Harbor, Me.); Fisher (Fisher Scientific, Pittsburgh, Pa.); Pharmingen (Pharmingen, San Diego, Calif.); Santa Cruz Biotechnology (CA); Qiagen (Qiagen, Valencia, Calif.); Life Technologies (Life Technologies, Rockville, Md.); and UCSF (University of California, San Francisco, Calif.).

### EXAMPLE 1

### Cell Culture and Primary Myoblast Isolation

Actively growing C2C12, Sol8, and L6 skeletal myoblasts (ATCC) were maintained in DMEM with 10%, 20%, or 10% FBS, respectively. QM7 quail skeletal myoblasts (kindly provided by Charles Ordahl, UCSF) were maintained in M199 medium with 10% tryptose phosphate and 10% FBS. Myoblast differentiation was induced by culture of confluent monolayers in DMEM with 1% FBS for C2C12 and Sol8 cells, 2% horse serum for L6 cells, or M199 medium with 10% tryptase phosphate and 0.5% FBS for QM7 cells. For PIPLC treatment, 5 U/ml of PIPLC (Sigma) was added into the culture medium when cells reached confluence, and with daily media changes. For antibody treatments, 5 μg/ml monoclonal anti-Sca-1 or anti-CD34 were added to medium when cells reached confluence, and with medium changes. Monoclonal anti-Sca-1 antibodies, clones E13-161.7 and D7, were obtained from Pharmingen. Monoclonal anti-CD34 antibody was obtained from Santa Cruz Biotechnology.

Primary skeletal myoblasts were isolated according to standard procedures (See, e.g., Blanco-Bose et al., Exp Cell Res, 265:212-220, 2001). Skeletal muscle harvested from the hind limbs of 10-20 C57BL/6 neonatal (3 day old) mice was minced in cold, sterile phosphate-buffered saline (PBS), digested with 1.5 U/ml collagenase D, 2.4 U/ml dispase II, 2.5 mM CaCl₂ at 37° C., filtered through 40 μm nylon mesh, centrifuged, resuspended in 80% Ham's F-10 nutrient mixture, 20% FBS, 2.5 ng/ml basic fibroblast growth factor (bFGF), and plated on laminin-coated dishes. After 24 hours, cells were harvested with trypsin-EDTA, incubated with monoclonal rat anti-α7 integrin antibody, clone CY8 obtained from R. Kramer, UCSF (Yao et al., J Biol Chem, 271:25598-25603, 1996), selected at least 5 times with MACS goat anti-rat IgG MicroBeads on an autoMACS magnetic cell sorter (Miltenyi Biotec), and re-plated in 40% Ham's F-10 nutrient mixture, 40% Dulbecco's Modified Eagle's media, 20% FBS, 2.5 ng/ml bFGF (proliferation medium) on collagen-coated dishes. Purity was analyzed by flow cytometric detection of α7 integrin, using monoclonal rat anti-α7 integrin antibody and phycoerythrin (PE)-conjugated donkey anti-rat F(ab′)2 fragment (Jackson ImmunoResearch). Sorted cells were grown in proliferation medium for 5 days, and then transferred to differentiation medium (80% Dulbecco's Modified Eagle's media, 2% horse serum) to stimulate myotube formation.

### EXAMPLE 2

### BrdU Incorporation and Myoblast Fusion Assays

Cells were plated on cover slips, cultured under conditions described above in Example 1, and labeled with BrdU for 60 min using Labeling and Detection Kit I (Roche) according to the manufacturer's instructions. Cells were stained with a 1:10 dilution monoclonal anti-BrdU (Roche), followed by 4 μg/ml Alexa Fluor 594-conjugated goat anti-mouse IgG in incubation buffer provided with the Labeling and Detection Kit I. Cells were co-stained with antibodies against Myf5, MyoD, or myogenin as described in Example 6. Prior to mounting cover slips on slides, cells were incubated with 1 μg/ml DAPI (Sigma) for 5 min at room temperature. Immunofluorescence signals were acquired with a Nikon Microphot-FX fluorescence microscope and Spot imaging software. Nuclei from low-power (10×) images were analyzed to quantitate BrdU incorporation and myoblast fusion. Each culture stage was analyzed in triplicate, with data representing the mean of 10 high-power fields. Cell proliferation was calculated as percent BrdU-positive nuclei per field. Cell fusion was calculated as average number of DAPI staining nuclei per myotube.

### EXAMPLE 3

### Blocking SCA-1 Function by PIPLC Treatment

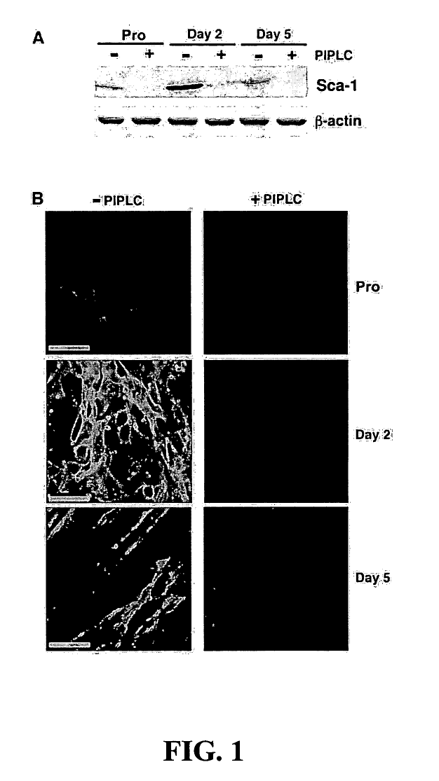

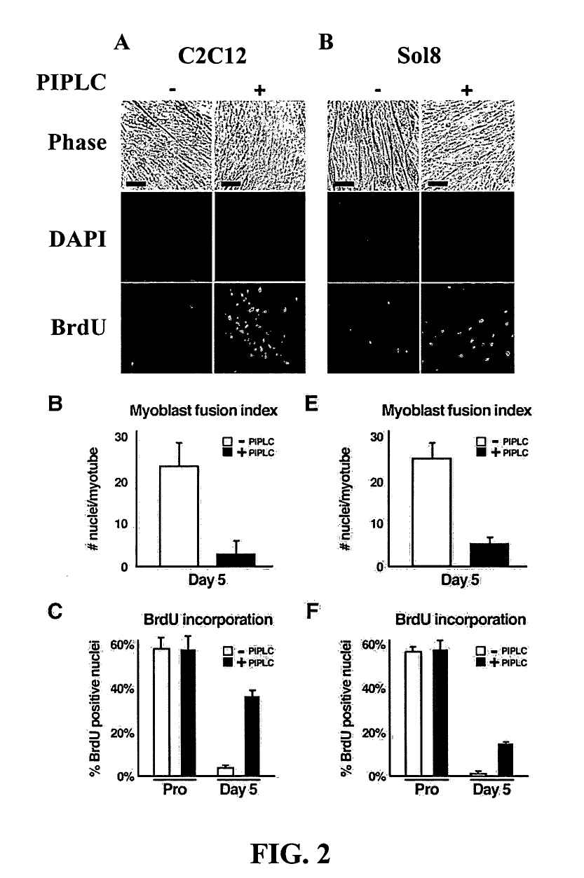

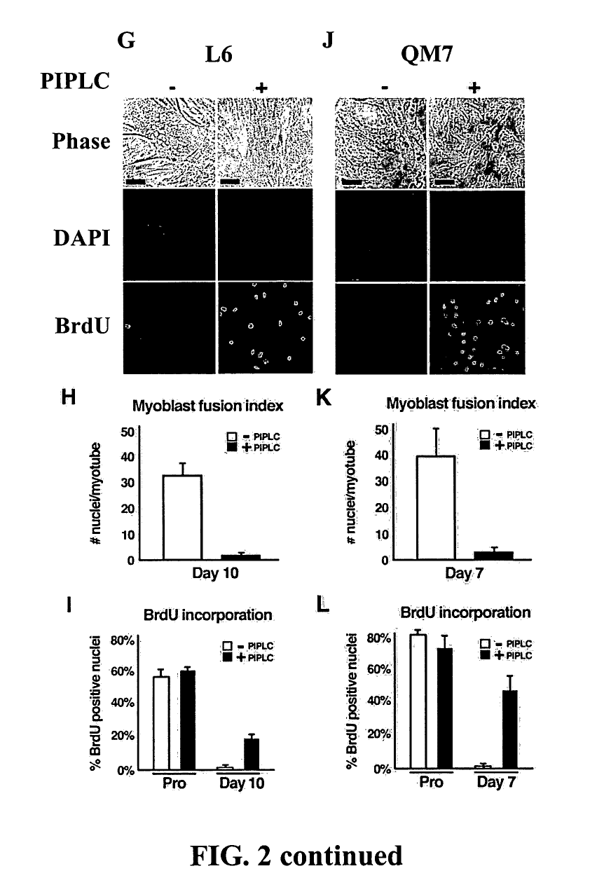

This example describes the use of PIPLC (Griffith and Ryan, Biochim Biophys Acta, 1441:237-254, 1999) to strip GPI-anchored proteins from the surface of C2C12 myoblasts, to investigate the role of GPI-anchored cell surface proteins in myoblast differentiation, as shown in FIG. 1. Treatment with PIPLC had a profound effect on myotube formation (FIG. 2), yielding a population of cells primarily composed of dividing, mononuclear myoblasts even when grown under differentiation conditions. Specifically, as shown in FIG. 2, removal of GPI-anchored proteins from the surface of C2C12 myoblasts resulted in a decrease in myoblast fusion (3±1 versus 23±5 nuclei/myotube) and an increase in BrdU incorporation (37±2% versus 3±1% BrdU positive nuclei), under conditions that normally would produce terminally differentiated myotubes (Shen et al., Dev Dyn, 226, 128-138, 2003).

Similar experiments were completed using Sol8 murine myoblasts (FIG. 2D-F), L6 rat myoblasts (FIG. 2G-I), and QM7 avian myoblasts (FIG. 2J-L). Consistent with the findings in murine C2C12 cells, PIPLC decreased myoblast fusion and increased BrdU incorporation in Sol8 (5±1 versus 25±3 nuclei/myotube; 18±2% versus 1±1% BrdU positive nuclei), L6 (2±1 versus 32±5 nuclei/myotube; 20±2% versus 2±1% BrdU positive nuclei), and QM7 (1±0.2 versus 40±10 nuclei/myotube; 48±5% versus 1±0.5% BrdU positive nuclei) cells. Thus, GPI-anchored protein(s), such as Sca-1, were shown to play a general role in regulating cell cycle withdrawal and myotube formation during myocyte differentiation.

### EXAMPLE 4

### Blocking SCA-1 Function by Antibody Treatment

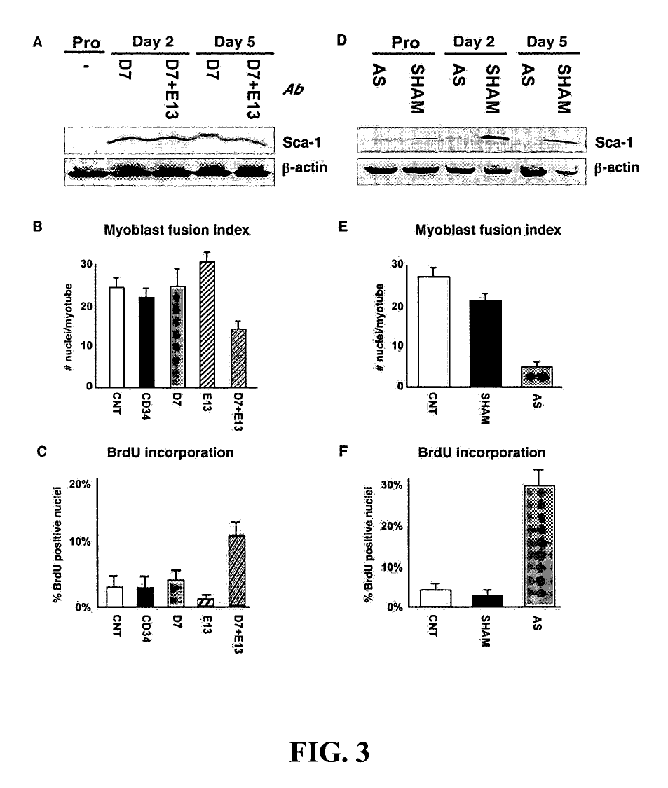

Anti-Sca-1 blocking antibodies have been used to inhibit aggregation of thymocytes expressing Sca-1 on their surfaces (Bamezai and Rock, Proc Natl Acad Sci USA, 92:4294-4298, 1995; and English et al., J Immunol, 165:3763-3771, 2000). To examine directly the role of Sca-1 in myoblast fusion and cell cycle withdrawal, C2C12 cells were cultured in the presence of anti-Sca-1 antibodies. Clone E13-161.7 was raised against BALB/c mouse “pre-T” cells (Aihara et al., Eur J Immunol, 16:1391-1399, 1986), while clone D7 was raised against the IL-2-dependent mouse T-cell line CTL-L (Ortega et al., J Immunol, 137:3240-3246, 1986). Although both antibodies react with Sca-1, they recognize distinct epitopes since D7 is unable to block binding of E13-161.7 (Bamezai and Rock, supra, 1995). As observed during development of the present invention, treatment with either antibody alone produced no significant difference in myoblast fusion or DNA synthesis, compared with untreated cells. In contrast as shown in FIG. 3, panels A-C, treatment with the two antibodies in combination, blocked fusion and sustained myoblast proliferation, similar to the effects seen with PIPLC treatment (14±2 versus 25±7 nuclei per myotube; 11±3% versus 3±1% BrdU positive nuclei). These effects on myoblast fusion and DNA synthesis could be seen as far out as at 10 days of antibody treatment, with complete reversal of these effects observed upon removal of antibody.

Previously, it has been shown that the D7 antibody alone can inhibit thymocyte aggregation by 80-90% (Bamezai and Rock, supra, 1995), however, during development of the present invention, D7 was unable to block myoblast fusion in the absence of E13-161.7 antibody. Thus, the mechanism for Sca-1-mediated cell fusion of myoblasts apparently differs from its mechanism for aggregation in thymocytes. Even so, an understanding of any of the mechanism(s) involved is not necessary in order to make or use the present invention, and it is not intended that the present invention be limited to any particular mechanism(s).

### EXAMPLE 5

### Blocking SCA-1 Function by Antisense Expression

This example describes the generation of stable antisense Sca-1 transfectants of C2C12 cells. Briefly, a 330 bp fragment encoding nucleotides −9 to +321 of the Sca-1 coding sequence (Stanford et al., Immunogenetics, 35:408-411, 1992), and flanked by EcoRI sites, was cloned by PCR from total mouse cDNA, inserted into the EcoRI site of vector pcDNA3.1A (Invitrogen), and screened for antisense insert orientation. The newly generated pLy6AS or empty pcDNA3.1A vectors were transfected into C2C12 cells using the LipofectAMINE Plus reagent (Invitrogen) as instructed by the manufacturer. After 10 days of incubation in medium containing 800 μg/ml G418 (Life Technologies), resistant clones were isolated, expanded, and screened for attenuation of Sca-1 expression by immunostaining and immunoblot, as described in Example 6. Stable cell lines were maintained in culture medium with 500 μg/ml G418. Experiments were performed in multiple clones.

Although stable sense Sca1 transfectants were also made, Sca-1-overexpressing cells were found to behaved no differently than sham-transfected or untransfected cells, and were not included in subsequent experiments. Antisense-expressing cells showed a significant downregulation of Sca-1 expression, compared with a cell line stably transfected with empty vector (FIG. 3D and FIG. 10B). When grown in differentiation media, Sca-1 antisense-expressing cells displayed a fusion defect with short, paucinuclear myotubes (fusion index 0.09±0.07 versus 0.65±0.05; P<0.001) as shown in FIG. 3E, and retained a higher proliferation index (29±4% versus 3±1% BrdU-positive nuclei; P<0.001) as shown in FIG. 3F, compared with untransfected or sham-transfected cells. As demonstrated herein, Sca-1 is necessary for proper cell-cycle withdrawal and myotube formation during C2C12 differentiation.

Because others have shown that myoblasts that fail to differentiate undergo apoptosis, the inventors also analyzed Sca-1 antisense-expressing cells for cell death and p21 expression. Compared with control C2C12 cells, which showed appropriate induction of p21 expression and protection against apoptosis during differentiation, Sca-1 antisense expressing cells continued to proliferate in differentiation medium, failed to induce p21 expression, and demonstrated an increase in apoptosis. Thus, the inventors contemplate that Sca-1 influences p21 expression, and consequently protection against apoptosis. Nonetheless, an understanding of any of the mechanism(s) involved is not necessary in order to make or use the present invention, and it is not intended that the present invention be limited to any particular mechanism(s).

### EXAMPLE 6

### Immunoblot and Immunophenotype Analysis

Immunoblot analysis was performed using previously described methods (Hlaing et al., J Biol Chem, 277:23794-23799, 2002). Briefly, cells were lysed by sonication in lysis buffer (50 mM Tris-HCl, pH 7.5, 150 mM NaCl, 3 mM EDTA, 3 mM EGTA, 1% SDS, 10% glycerol), boiled for 5 min, and centrifuged at 16,000×g for 2 min. Protein concentrations of supernatants were measured by BCA assay (Pierce), and equal amounts of protein (100-200 μg) were separated by SDS-polyacrylamide gel electrophoresis. Immunoblots were analyzed with 5 μg/ml monoclonal antibody against Sca-1 (Pharmingen) or β-actin (Sigma), and 1 μg/ml HRP-conjugated goat-anti mouse IgG (Bio-Rad).

To detect Sca-1, Myf5, MyoD, and myogenin in situ, wild type and stably transfected C2C12 cells were plated on cover slips, grown as described for the indicated lengths of time, and stained with either 2.5 μg/ml monoclonal anti-Sca-1 antibody (clone E13-161.7; Pharmingen) and 4 μg/ml FITC-conjugated goat anti-rat IgG (Pharmingen), or 10 μg/ml rabbit polyclonal anti-Myf5, anti-MyoD, or anti-myogenin antibody (Santa Cruz Biotechnology) and 2 μg/ml FITC-conjugated goat anti-rabbit IgG (Molecular Probes), as previously described (Liu et al., Cell Biochem Biophys, 39:119-131, 2003).

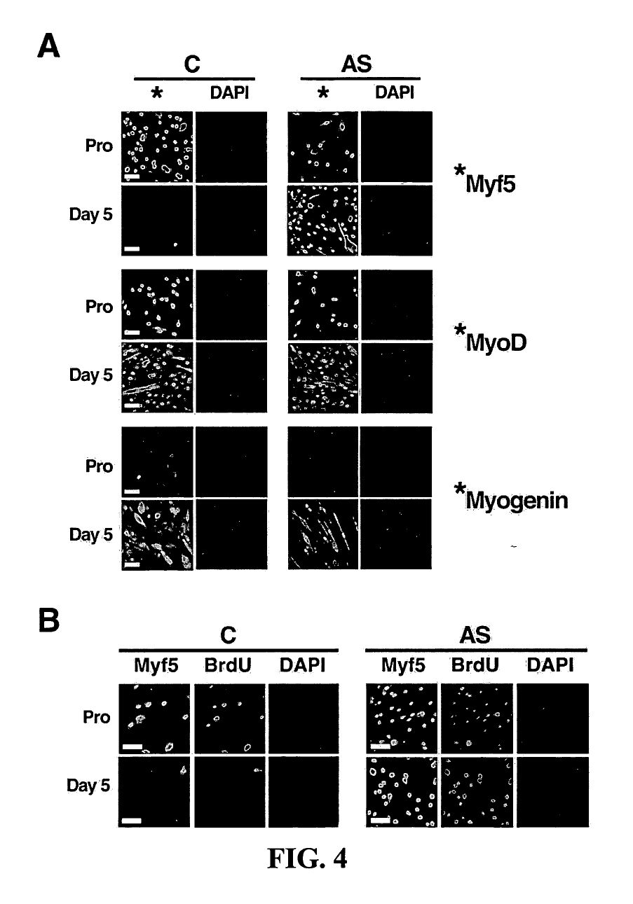

Briefly, as shown in FIG. 4 panel A, C2C12 cells expressing Sca-1 antisense were grown in differentiation medium for five days and co-stained with antibodies against Myf5, MyoD, and myogenin, as markers of specific stages of C2C12 differentiation (Sabourin and Rudnicki, Clin Genet, 57:16-25, 2000; and Shimokawa et al., Biochem Biophys Res Commun, 246:287-292, 1998). Myf5 and MyoD staining was observed in proliferating control and antisense cells, while MyoD and myogenin staining was observed in differentiated control and antisense cells. Expression of the early marker, Myf5, however, persisted in Sca-1 antisense cells grown under differentiation conditions.

To determine whether the Myf5-positive, Sca-1 antisense cells grown under differentiating conditions comprised the pool of proliferating cells previously detected, the cells were labeled with BrdU, and co-stained with antibodies against BrdU and Myf5. As shown in FIG. 4 panel B, the majority of Myf5-positive Sca-1 antisense cells were actively proliferating. Thus, inhibition of Sca-1 expression results in a proliferating pool of mononuclear myoblasts that persist in an early stage of differentiation, despite culture under conditions suitable for myotube formation in control cells.

### EXAMPLE 7

### FYN and SRC Kinase Assays

Some GPI-anchored proteins signal through Src family tyrosine kinases (Horejsi et al., Immunol Lett, 63:63-73, 1998; Marmor and Julius, J Biol Regul Homeost Agents, 14:99-115, 2000). Specifically, Sca-1/Ly-6A has been shown to signal through Fyn during T-cell activation in vitro (Lee et al., EMBO J, 13:2167-2176, 1994). However, while in vitro crosslinking of Sca-1 in T cells results in Fyn autophosphorylation, observations with Sca-1 null mice suggest that Sca-1 downmodulates lymphocyte responses (Stanford et al., J Exp Med, 186:705-717, 1997). More recently, Fyn activity has been implicated in the regulation of anti-apoptotic pathways in differentiated myotubes, but specifically has been shown to be inactive during earlier stages of myogenesis (Laprise et al., J Cell Physiol, 191:69-81, 2002). For this reason, experiments were conducted during development of the present invention, to determine whether Sca-1 function in myoblasts is mediated through Fyn and/or Src in myoblasts stimulated to form myotubes.

Briefly, cells were cultured under conditions described above in Example 1, lysed in lysis buffer (25 mM Tris, pH 7.2, 150 mM NaCl, 100 μm Na₃VO₄, 1% NP-40, 10 mM NaF, 1:1000 Sigma Protease inhibitor cocktail) for 30 min on ice, sheered by needle passage, then centrifuged at 11,750×g for 6 min at 4° C. Total supernatant protein was determined by BCA assay (Pierce). One mg total protein was incubated with 2 μg monoclonal anti-Fyn or anti-Src antibody (Santa Cruz Biotechnology) for 2 hr at 4° C., and 50 μl protein-A sepharose (Sigma) for an additional 30 min. Immune complexes were washed with ice-cold lysis buffer, then re-suspended in ice-cold 40 μl kinase reaction buffer (100 mM Tris pH 7.2, 125 mM MgCl₂, 25 mM MnCl₂, 2 mM EGTA, 25 μM Na₃VO₄, 2 mM DTT) containing 20 μM ATP and 4 μM γ(P³²)ATP (6000 mCi/mmol). Reactions were incubated with 150 μmol substrate peptide from Upstate ([K19] cdc2 (6-20)-NH2, KVEKIGEGTYGVVKK; SEQ ID NO:6) for 10 min at 30° C. with vigorous agitation. Reactions were stopped on ice and proteins precipitated with 20 μl 40% tricholoracetic acid. Precipitates were collected on phosphocellulose squares (Upstate), washed with 0.75% phosphoric acid then acetone, and dried prior to measuring incorporated radioactivity by Cerenkov counting. Counts were normalized against immunoprecipitated Fyn or Src protein determined by immunoblot with monoclonal anti-Fyn or anti-Src antibody and densitometry (Adobe Photoshop and NIH/Object-Image), and expressed as fold-increase over background (immunoprecipitation performed without primary anti-Fyn or anti-Src antibody).

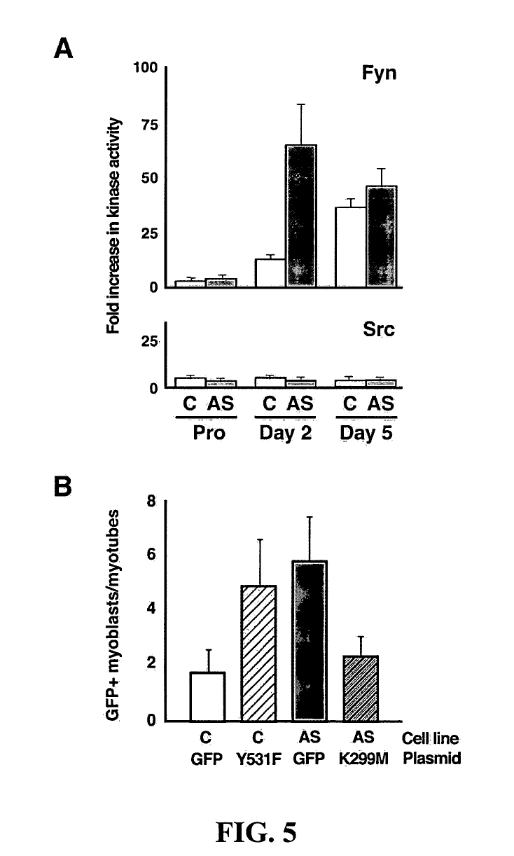

As shown in FIG. 5 panel A, both wild type and Sca-1 antisense cells demonstrated constitutive Src activity, independent of differentiation stage or Sca-1 expression. In contrast, Fyn activity was elevated in wild type cells at day 5 in differentiation medium. Moreover, Fyn activity was elevated in Sca-1 antisense myoblasts at day 2 in differentiation medium, as compared to wild type cells. This elevation in Fun activity coincides with the transient increase in Sca-1 expression observed during myoblast cell cycle withdrawal and early differentiation. Thus the inventors contemplate that inappropriate Fyn activity at an earlier stage of myogenesis provides a mechanism for the sustained proliferation and arrest in myogenic differentiation seen in Sca-1 antisense cells. Even so, an understanding of any of the mechanism(s) involved is not necessary in order to make or use the present invention, and it is not intended that the present invention be limited to any particular mechanism(s).

### EXAMPLE 8

### FYN Mutant Assays

This example describes the expression of wild type and mutant Fyn proteins to analyse the role of Fyn in Sca-1-mediated myogenesis. Briefly, a 1615 bp fragment of pCMV-FynDN (Ko et al., J. Biol Chem, 277:46085-46092, 2002), containing the full-length, dominant negative K299M mutant of mouse Fyn, was PCR-amplified using primers 5′-TTATGCGGCC GCATGGGCTGTG TGCAATGTAAGG-3′ (SEQ ID NO:7) and 5′-GCATGCATGC TACAGGTTT TCACCAGGTT GG-3′ (SEQ ID NO:8) and cloned into the NotI/SphI sites of pIRES-hrGFP-1a (Stratagene). The pCMV-FynDN plasmid was kindly provided by Marilyn Resh (Sloan-Kettering Cancer Center). Similarly, a 1.6 kb BamHI fragment of Fyn-Y531F (Maulon et al., Oncogene, 20:1964-1972, 2001), containing the full-length, constitutively active Y531F mutant of mouse Fyn, was cloned into the BamHI site of pIRES-hrGFP-1a (Stratagene). The Fyn-Y531F DNA was generously provided by Patrick Auberger (INSERM). Sequences for both plasmids expressing Fyn mutants and GFP were confirmed. Expression of mutants and GFP was determined by immunoblot of whole cell lysates from transfected C2C12 cells with anti-Fyn antibody (Santa Cruz Biotechnology), and immunofluorescence detection of GFP in live cells. Cells were plated on cover slips and cultured as described above. Transfections were performed using Lipofectamine 2000 (Invitrogen) according to the manufacturer's instructions, 24 hours prior to switching cultures to differentiation conditions. Prior to mounting cover slips on slides, cells were incubated with 1 μg/ml DAPI (Sigma) for 5 min at room temperature. Data was acquired with a Nikon Microphot-FX fluorescence microscope and Spot imaging software. Each set of transfections with mutant Fyn constructs was performed in triplicate, with data representing the mean of 10 medium-power (20×) fields (>200 nuclei per field). The effect of Fyn mutants on cell fusion was measured as the ratio of GFP-positive mononuclear myoblasts to GFP-positive myotubes (cells with 2 or more nuclei).

In the first place, a dominant-negative mutant of Fyn was co-expressed with GFP in Sca-1 antisense-expressing cells. As shown in FIG. 5 panel B, the ratio of transfected myoblasts to myotubes (as indicated by GFP expression) after five days in differentiation medium was found to be lower in cells expressing the dominant-negative mutant Fyn as compared to cells expressing GFP alone (2.3+/−0.7 vs. 5.7+/−1.6). Conversely, a constitutively active form of Fyn was co-expressed with GFP in wild type C2C12 cell. As also shown in FIG. 5 panel B, the ratio of transfected myoblasts to myotubes at five days in differentiation medium was found to be higher in cells expressing constitutively active Fyn as compared to cells expressing GFP alone (4.9+/−1.7 vs. 1.7+/−0.8). These studies demonstrated that dominant negative Fyn rescued the Sca-1-antisense phenotype, while active Fyn produced the Sca-1-antisense phenotype in wild type C2C12 cells. Thus, as described for the first time herein, the effects of Sca-1 in differentiating myoblasts were found to be mediated at least in part by Fyn, Sca-1 expression is coupled to the downregulation of Fyn activity during early myogenesis, and inappropriate activation of Fyn is associated with sustained myoblast proliferation and a defect in myoblast fusion. Nonetheless, an understanding of any of the mechanism(s) involved is not necessary in order to make or use the present invention, and it is not intended that the present invention be limited to any particular mechanism(s).

### EXAMPLE 9

### Anatomic and Histologic Analysis of SCA-1−/− Muscle

The structure and organization of skeletal and cardiac muscle from Sca-1^{-/-} mice is examined as follows. A colony of Sca-1-null mice is established from C57BL/6 backcrossed breeding pairs generously provided by Patrick Flood (University of North Carolina), whose lab generated the mice (Stanford et al., J Exp Med, 186:705-717, 1997). The hearts and skeletal muscle of Sca-1-null mice are examined at 2 days, 3 weeks (by which time spontaneous cardiac myocyte proliferation has effectively ceased in mice as previously described by MacLellan and Schneider, Annu Rev Physiol, 62:289-319, 2000), 3 months, and 6 months of age.

In the first place, Sca-1-null hearts are examined for gross anatomical defects. To further evaluate the muscle from these animals, fresh hearts and hindlimb muscle from neonatal and adult Sca-1^{+/+}, Sca-1^{+/-} Sca-1^{-/-} animals are weighed and measure as a function of body weight. Muscle tissue samples are frozen in liquid nitrogen-cooled isopentane, to avoid fiber damage from imperfect dehydration and rehydration, and survey tissue sections are stained with hematoxylin-eosin. To assess myocyte number, frozen tissue sections are fixed in acetone and stained with wheat germ agglutinin to visualize myocyte borders (Dolber et al., J Mol Cell Cardiol, 24:1443-1457, 1992). To evaluate myofibrillar organization, frozen tissue sections are stained for f-actin with rhodamine-phalloidin. In addition, fibrosis as an indicator of muscle damage is assessed by fixing tissues in 4% paraformaldehyde, embedding the tissues in paraffin, and staining them with modified Masson's trichrome stain. For further ultrastructural analysis, tissue sections are fixed in 3% gluteraldehyde, 0.2% tannic acid in MOPS, pH 6.8, and myocyte cytoarchitecture and myofibrillar organization are examined by transmission electron microscopy.

### EXAMPLE 10

### Cell Cycle Profiling of SCA-1−/− Myocytes

The cell cycle behavior of Sca-1^{-/-} primary skeletal myoblasts and cardiac myocytes, as compared with cells from Sca-1^{+/-} and Sca-1^{+/+} animals are examined as follows. Primary myoblasts are isolated using standard procedures known in the art (Blanco-Bose et al., Exp Cell Res, 265:212-220, 2001). Briefly, muscle tissue is harvested from the hindlimbs of 10 to 20 neonatal (3 days old) mice in cold, sterile phosphate-buffered saline. Minced muscle tissue is digested with 1.5 U/ml collagenase D and 2.4 U/ml dispase II, in a buffer containing 2.5 mM CaCl₂, for 90-120 minutes at 37° C. The digested muscle tissue is filtered through 80 μm nylon mesh, centrifuged, and then resuspend in media that provides a growth advantage to myoblasts over fibroblasts (80% Ham's F-10 nutrient mixture containing 20% FBS, and 2.5 ng/ml basic fibroblast growth factor. The resulting cells are then plated on laminin-coated dishes. In addition, cardiac myocytes are isolated using procedures the inventors have adapted from standard methods (Deng et al., Circ Res, 87:781-788, 2000). Briefly, single cell suspensions from neonatal (3 day-old) mouse hearts are isolated by mechanical dissociation and enzymatic digestion. This procedure has been modified to include an enzymatic dissociation step with collagenase D (Roche Molecular Biochemical) and with DNAase II as reported by others (Soonpaa et al., Am J Physiol, 271:H2183-2189, 1996).

The percentage of cycling myoblast-derived cells is determined by flow cytometry (Bernstein and Coughlin, J Biol Chem, 273:4666-4671, 1998; and Hlaing et al., J Biol Chem, 277:23794-23799, 2002). Cells are fixed in 4% paraformaldehyde and permeabilized with 0.1% Triton X-100. Myocytes are distinguished from non-muscle cells by staining with a monoclonal antibody (clone MF20) against striated muscle myosin heavy chain (Developmental Studies Hybridoma Bank) and FITC-conjugated goat anti-mouse IgG (Sigma). Sca-1+ cells are labeled with a monoclonal anti-Sca-1 antibody produced by the E13 clone (Pharmingen) and PE-conjugated donkey anti-rat F(ab′)2 (Jackson Immuno Research). DNA is labeled with propidium iodide. Propidium iodide, FITC, and PE signals are acquired with a Becton Dickinson FACStar Plus with dual argon-ion lasers at 488 nm and 363 nm light output and with 630/22 mm, 530/30 mm, and 575/20-mm bandpass filters, respectively. The DNA content from at least 20,000 events (cells) is analyzed using CellQuest software (Becton Dickinson). A shift in the percentage of Sca-1^{-/-} or Sca-1^{+/-} cells with greater than 2N DNA content (S-phase or G2/M), from either MF20-positive or −negative populations, compared with Sca-1^{+/+} cells, is contemplated to support the role of Sca-1 in regulating cell cycle withdrawal in vivo in this model.

### EXAMPLE 11

### Fusion Capacity of SCA-1−/− Myocytes

The fusion capacity of Sca-1^{-/-} and Sca-1^{+/-} cells isolated as described in Example 10, is assessed by culturing them under differentiation conditions, and monitoring them for fusion events, both with respect to the time it takes to observe cells with more than one nucleus, and the extent of fusion as a function of number of myotubes per total number of nuclei and number of nuclei per myotube. Briefly, cells are immunostained for sarcomeric myosin using a polyclonal anti-myosin (Sigma), while nuclei are stained with DAPI. The number of myosin-positive cells with 2 or more nuclei/total number of nuclei (fusion index) is determined as described in Example 2. Alternatively, to achieve a high-throughput survey of early fusion events, adherent cells are returned to suspension and initial fusion events are assessed as a function of size using a Coulter Multisizer counter (Beckman). This technique passes cells through an aperture, where an electric field is distorted in a size-dependent manner, providing a size distribution for cell populations. This technique has been used to distinguish small changes in cardiomyocyte volume in response to hypertrophic stimuli (Irlbeck et al., J Mol Cell Cardiol, 29:2931-2939, 1997), and is contemplated to provide a more sensitive way to detect initial fusion events. A decrease in the number and/or quality of fusion events in Sca-1-null cells is contemplated to support a role of Sca-1 in mediating myoblast fusion in vivo. Specifically, a decrease in the number of cells with more than one nucleus indicates a role for Sca-1 in fusion initiation, while a decrease in the average number of nuclei per myotube indicates a role for Sca-1 in subsequent, secondary fusion events. Nonetheless, an understanding of any of the mechanism(s) involved is not necessary in order to make or use the present invention, and it is not intended that the present invention be limited to any particular mechanism(s).

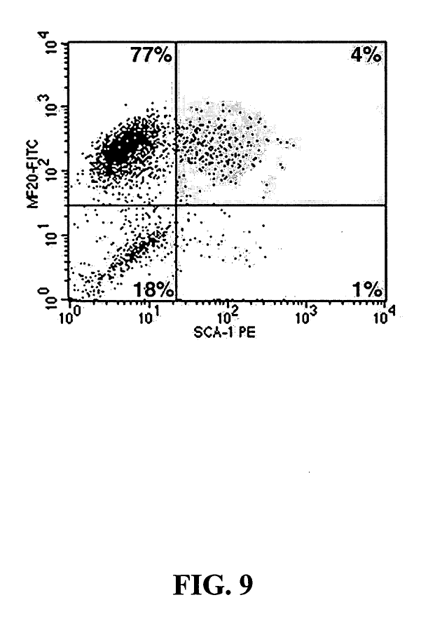

Binucleated cardiac myocytes are presumed to result from nuclear division without cytokinesis (Oh et al., Proc Natl Acad Sci USA, 100:12313-12318, 2003). However, as described herein, the presence of Sca-1 on a subpopulation of cardiac myocytes (FIG. 9) and the evidence of a role for Sca-1 in myoblast fusion, indicates that cardiac myocyte binucleation also occurs through cell fusion. The ratio of mononuclear to binuclear cardiac myocytes in Sca-1^{-/-} and Sca-1^{+/-} mice, as compared with Sca-1^{+/+} animals, is determined with either flow cytometry or Coulter Multisizer counting. Flow cytometry permits the sorting of cells with 4N DNA content as described in Example 10, for subsequent direct examination of the sorted cells for the presence of one (G2 phase or mitotic) or two nuclei. In particular, single cell suspensions of cardiac cells are stained with MF20 (to determine muscle versus non-muscle cells) and propidium iodide (to determine DNA content), and MF20⁺ cells with 4N DNA content are selected. The MF20+4N DNA cells are plated on microscope slides, and stained with hematoxylin-eosin, for determination of the number of mononuclear and binuclear cells by direct visualization of at least 500 cells per slide. Alternatively, Coulter Multisizer counting permits these two cell populations to be distinguished on the basis of cell size.

### EXAMPLE 12

### Repair Response to Muscle Injury In Vivo

To investigate the role of Sca-1 in muscle injury repair, myonecrotic injuries are induced in pairs of 2-day, 4- and 8-week old Sca-1^{+/+}, Sca-1^{+/-}, and Sca-1^{-/-} mice by using notexin, a myotoxic phospholipase isolated from the Australian tiger snake (Notechis scutatus) as described (Harris and Cullen, Electron Microsc Rev, 3:183-211, 1990). A toxic injury model is selected since this method demonstrates greater conservation of the basement membrane of necrotic myofibers, and earlier differentiation and proliferation of muscle precursor cells, compared with ischemic injury and dennervation (Lefaucheur and Sebille, Neuromuscul Disord, 5:501-509, 1995). Briefly, the tibialis anterior muscle on the hindlimbs of appropriately anesthetized animals is exposed, for injection in the right hindlimb muscle with 0.5 μg notexin (Sigma) in 10 μl PBS and the left muscle with 10 μl PBS as an injection control. Animals are sacrificed at 4, 7, 10, and 14 days after injection, and muscle injury and repair is analyzed by anatomic and histologic methods as described in Example 9. Delayed repair, increased scarring, or myofibrillar disarray in Sca-1-deficient muscle injected with notexin versus saline, is contemplated to support a role for Sca-1 in events leading to skeletal muscle regeneration.

### EXAMPLE 13

### Expansion of Isolated Cells In Vitro

Primary murine skeletal myoblasts or cardiac myocytes are isolated, resuspended in ice-cold PBS, and then stained with an anti-Sca-1 mAb as described in Example 10. Alternatively, murine Sca-1+ stem cells are isolated from peripheral blood or bone marrow using art-recognized methods (See e.g., U.S. Pat. No. 5,087,570 to Weismann et al., herein incorporated by reference). Sca-1+ cells are then selected with either FACS or with MACS goat anti-rat IgG MicroBeads on an autoMACS magnetic cell sorter (Miltnyi Biotec).

The sorted cells are plated in medium containing 40% Ham's F-10 nutrient mixture, 40% DMEM, 20% FBS, and 2.5 ng/ml basic fibroblast growth factor (proliferation medium) on collagen-coated plates in the presence of a Sca-1 antagonist (PIPLC, anti-Sca-1 antibody combination and/or Sca-1 antisense). The collagen-coated plates are produced by coating 100 mm dishes with 4 ml of solution made from mixing 15 ml collagen (0.01%) and 10 ml PBS. The plates containing the mixture are rocked from 3 hours to overnight at RT, then 6 ml PBS is added directly into the mixture. The plates containing 10 ml collagen in PBS are stored at 4° C. for up to 2 weeks. Just prior to use, liquid is aspirated from the plates without creating dry areas, and cells are directly plated onto the wet surfaces.

In other embodiments, after growing the sorted cells in proliferation medium for 5 days, the cells are transferred to differentiation medium (80% DMEM, 2% horse serum) in the presence of a Sca-1 antagonist to stimulate muscle development.

### EXAMPLE 14

### Transplantation of Expanded Myocytes for Myocardial Regeneration

This example describes the testing of the expanded cells populations of Example 13, in a murine model of myocardial infarction. Myocardial infarction is induced in female C57BL/g mice at about 2 months of age as described (Li et al., J Clin Invest, 100:1991-1999, 1997). Briefly, under anesthesia, the thorax of recipient mice is opened, the heart is exteriorized, and the left main coronary artery is ligated. The chest is then closed, the pneumothorax reduced, and the mice are allowed to recover. Control mice undergo a sham operation (no ligation). Approximately 3 to 5 hrs after infarction, the thorax is re-opened and 2.5 ml PBS containing either freshly-isolated or expanded cells from male mice are injected into the anterior and posterior aspects of the viable myocardium bordering the infarct. Approximately, 1, 2 and 4 weeks after transplantation, recipient and control mice are sacrificed. Ventricular function and Y chromosome staining of the harvested hearts are assessed as described (See e.g., Orlic et al., Nature, 410:701-705, 2001; and U.S. Patent Application Publication No. 20020098167 by Anversa et al; herein incorporated by reference in their entirety). Mice receiving the Sca-1+ selected cells expanded in the presence of a Sca-1 antagonist, are contemplated to have superior ventricular function and greater Y chromosome staining (greater engraftment), as compared to mice receiving unselected or unexpanded cell populations.

### EXAMPLE 15

### Transplantation of Expanded Myocytes for Repair of Skeletal Muscle

This example describes the testing of the expanded cells populations of Example 13, in the mdx mouse model of Duchenne's muscular dystrophy (Sicinski et al., Science, 244:1578-1580, 1989). Both C57BL/10 and C57BL/10ScSn-Dmd^{mdx}/J (X-linked muscular dystrophy) mice are obtained from the Jackson Laboratory. Briefly, freshly isolated and expanded cells (containing a wild type dystrophin gene) obtained from normal male mice are washed, resuspended in PBS, and administered by tail vein injection (IV) or by intramuscular (IM) injection into the tibialis anterior (TA) muscle of female mdx mice. After 4, 8 and 12 weeks post cell transplantation, the TA muscles from the transplant recipients are analysed for dystrophin expression by immunohistochemistry and fluorescence in situ hybridization (FISH) analysis using a Y-chromosome probe to detect donor-derived (male) cells as described (See e.g., Gussoni et al., Nature, 401:390-394, 1999; and U.S. Patent Application Publication Nos. 20020182192 and 20030003085 by Kunkel et al; herein incorporated by reference in their entirety). Mice receiving the Sca-1+ selected cells expanded in the presence of a Sca-1 antagonist, are contemplated to have greater dystrophin expression in myofibres (greater engraftment), as compared to mice receiving unselected or unexpanded cell populations.

### EXAMPLE 16

### Flow Cytometric Analysis

Briefly, C2C12 cells and primary skeletal myoblasts were detached from culture with 0.25% trypsin, 2 mM EDTA at indicated times. Cells were washed with PBS, 10% FBS and fixed with 0.4% paraformaldehyde for 30 minutes at room temperature. After fixation, cells were washed with PBS, 2% calf serum (wash buffer) and then incubated in 100 μl wash buffer, 1% goat serum, 0.1% Triton X-100 containing primary antibodies at 4° C. overnight. Cells were then washed with wash buffer and incubated in 100 μl wash buffer, 2 μg/ml DNAase-free RNAse A (Sigma) containing secondary antibodies at 37° C. for 1 hour. Prior to flow cytometric analysis, cells were washed with wash buffer, filtered through 40 μm nylon mesh to exclude cell clumps and large myotubes, and incubated with 5 μg/ml propidium iodide (PI) at room temperature for 30 minutes. Primary/secondary antibody combinations used included monoclonal rat anti-Sca-1 (clone E13-161.7; BD Pharmingen) with PE-conjugated donkey anti-rat F(ab′)2 fragment (Jackson ImmunoResearch), and monoclonal mouse anti-desmin (DAKO), anti-myosin (MF20; University of Iowa Developmental Studies Hybridoma Bank), or anti-MyoD (BD Pharmingen) with FITC-conjugated goat anti-mouse IgG (Sigma).

A Becton Dickinson FACSCalibur with 488 nm argon-ion laser was used to acquire propidium iodide (PI), FITC and PE signals with 630/22 mm, 530/30 mm and 575/20 mm bandpass filters, respectively. Forward and side angle light scatter plots (as indicators of cell size and granularity), and PI, FITC and PE signals (to detect DNA content, desmin, myosin or MyoD, and Sca-1, respectively), were collected for at least 10,000 cells. PI area versus width plots were used to verify that the gating strategy employed included cell populations ranging from mononuclear cells with 2N DNA content to small, tetraploid myotubes containing 8N DNA content (Sharpless et al., Acta Cytol, 19:577-581, 1975) that expressed MyoD and myosin. Data were analyzed using CellQuest software (Becton Dickinson).

### EXAMPLE 17

### Statistical Analysis

To determine significance between two groups, comparisons between means were made using the Student's t-test. Multiple group comparisons were performed by one-way ANOVA with Student-Newman-Keuls' post hoc test. Trendlines were generated by bivariate regression analysis using the Pearson coefficient. SPSS version 11 for Macintosh (SPSS) was used for all tests, with a 0.05 level of confidence accepted for statistical significance.

In summary, the present invention provides numerous advances and advantages over the prior art, including methods and compositions for the producing muscle cells or progenitors suitable for transplantation. All publications and patents mentioned in the above specification are herein incorporated by reference. Various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the art, are intended to be within the scope of the present invention.

1. A method comprising:
a) contacting at least one cell expressing Sca-1 with a Sca-1 antagonist under growth conditions to produce an expanded cell population; and
b) administering said cell population to a subject to regenerate muscle.

2. The method of claim 1, wherein said growth conditions comprise conditions suitable for sustaining cell proliferation in the absence of cell fusion.

3. The method of claim 1, wherein said growth conditions comprise conditions suitable for retaining Myf5 expression.

4. The method of claim 1, wherein said growth conditions comprise conditions suitable for inducing myogenin expression.

5. The method of claim 1, wherein said growth conditions comprise conditions suitable for the transient elevation of Fyn activity.

6. The method of claim 1, wherein said Sca-1 antagonist comprises PIPLC.

7. The method of claim 1, wherein said Sca-1 antagonist comprises a Sca-1-reactive antibody combination.

8. The method of claim 7, wherein said Sca-1-reactive antibody combination comprises an antibody produced by a D7 clone and an antibody produced by an E13-161.7 clone.

9. The method of claim 1, wherein said Sca-1 antagonist comprises a Sca-1 antisense molecule.

10. The method of claim 1, wherein said at least one cell is a purified from a tissue sample selected from the group consisting of blood, bone marrow, and skeletal muscle.

11. The method of claim 1, wherein said at least one cell is derived from said subject.

12. The method of claim 11, wherein said subject is a mammal.

13. The method of claim 1, wherein said administering is accomplished with at least one of the group consisting of trans-coronary artery catheter (TCAC), intra-venous (IV) injection and intramuscular injection (IM).

14. The method of claim 1, wherein said subject is diagnosed with a cardiovascular disease selected from the group consisting of atherosclerosis, ischemia, hypertension, restenosis, angina pectoris, rheumatic heart disease, congenital cardiovascular defect, and arterial inflammation.

15. The method of claim 1, wherein said subject is diagnosed with a skeletal muscle injury or muscular degeneration.

16. A method comprising:
a) contacting at least one cell expressing Sca-1 with a Sca-1 antagonist under growth conditions to produce an expanded cell population; and
b) administering said cell population to a subject to study the regeneration of muscle.

17. A composition comprising an expanded cell population produced by contacting at least one cell expressing Sca-1 with a Sca-1 antagonist under growth conditions, and a buffer, wherein said growth conditions comprise conditions suitable for sustaining cell proliferation in the absence of cell fusion.

18. The composition of claim 17, wherein said expanded cell population comprises at least 1×10⁵ cells.

19. The composition of claim 17, wherein a majority of cells of said expanded cell population express one or more of Myf5, MyoD, and Myogenin.

20. The composition of claim 19, wherein said majority of cells comprises at least 50% of said expanded cell population.
